(19) United States
(12) Patent Application Publication
(10) Pub. No.: US 2026/0209154 A1
(43) Pub. Date: Jul. 23, 2026

(54) PROCESS FOR PREPARING COMPOUNDS DERIVED FROM 4-(3-ETHOXY-4-HYDROXYPHENYL)BUTAN-2-ONE

(71) Applicant: L'OREAL, PARIS (FR)

(72) Inventors: Patricio Guerreiro, Aulnay-Sous-Bois (FR); Thibaut Martin, Le Thillay (FR); Thierry Bordier, Aulnay-Sous-Bois (FR); Lucie Leseurre, Chevilly-Larue (FR)

(73) Assignee: L'OREAL, Paris (FR)

(21) Appl. No.: 19/143,765

(22) PCT Filed: Dec. 28, 2023
(86) PCT No.: PCT/EP2023/087913

§ 371 Date: Jun. 26, 2025

(30) Foreign Application Priority Data
Dec. 28, 2022 (FR) FR2214601

(51) Int. Cl. C07C 49/255; C07C 45/73; C07C 45/79

(52) U.S. Cl. CPC C07C 49/255; C07C 45/73; C07C 45/79

(57) ABSTRACT

The present invention relates to a process for preparing one or more compounds derived from 4-(3-ethoxy-4-hydroxyphenyl)butan-2-one corresponding to formula (I), as described below, and the optical isomers or geometrical isomers thereof, and also the salts thereof with alkaline agents, and/or the solvates thereof, such as the hydrates. The invention also relates to one or more particular compounds derived from 4-(3-ethoxy-4-hydroxyphenyl)butan-2-one corresponding to formula (I′) as defined below.

The present invention relates to a process for preparing one or more compounds derived from 4-(3-ethoxy-4-hydroxyphenyl)butan-2-one corresponding to formula (I), as described below, and the optical isomers or geometrical isomers thereof, and also the salts thereof with an organic or inorganic acid or base, and/or the solvates thereof, such as the hydrates.

The invention also relates to one or more particular compounds derived from 4-(3-ethoxy-4-hydroxyphenyl)butan-2-one corresponding to formula (I′) as defined below.

Microorganisms can survive and spread relatively easily in cosmetic, dermatological, pharmaceutical, and food products that do not contain preserving agents.

Preserving agents are thus regularly added to most industrial preparations intended to be stored or preserved in order to prevent microbial growth over time.

Microbial contamination during the production of an industrial product is common, even when the starting ingredients placed in said product are “clean”, i.e. free of contaminating microorganisms.

Water, for example, which is omnipresent in most cosmetic, pharmaceutical, dermatological or even nutraceutical products, must be free of contaminating microorganisms. Similarly, the other ingredients must also be screened for the presence of contaminating microorganisms. The cleanliness during the production of these industrial products, the processing of the contents and the filling of the containers must be scrupulously monitored in order to minimize, or even eliminate, the presence of contaminating microorganisms.

Despite these precautions, the microbial integrity of products of this type may require the presence of one or more preserving agents or preservatives that are compatible with the product and the stability of the composition. The products must allow neither growth nor viability of contaminating microorganisms.

In addition, maintaining cleanliness during the use of such products still remains problematic, notably when they are grasped, because fingers, applicators, for example cosmetic applicators, and even the ambient air are not sterile. Preserving agents thus prove necessary in order to reduce contamination with microorganisms by consumers during normal use.

As a general rule, pathogenic microorganisms must be absent from all products sold, notably cosmetic products (Kirk Othmer Encyclopedia, Cosmetics, Martin M. Rieger, Apr. 12, 2000). Over the years, preserving problems have also led to the introduction of preserving agents with a varied spectrum of action in order to combat resistant contaminating microorganisms.

The compound 4-(3-ethoxy-4-hydroxyphenyl)butan-2-one, also known as ethylzingerone (EZ), is a preserving agent commonly used in compositions, notably cosmetic compositions, for the advantageous performance thereof against various strains of bacteria, both Gram-positive bacteria and Gram-negative bacteria, on fungi and yeasts. Such a compound also exhibits high activity against bacteria of the Burkholderia cepacia complex.

The compound 4-(3-ethoxy-4-hydroxyphenyl)butan-2-one is generally used, alone or as a mixture, with other preservating or non-preserving compounds, to effectively protect various compositions, notably cosmetic compositions, from microbial contamination both during preparation and during the use thereof in daily life.

The compound 4-(3-ethoxy-4-hydroxyphenyl)butan-2-one may notably be prepared from ethylvanillin by the following synthetic pathway (A):

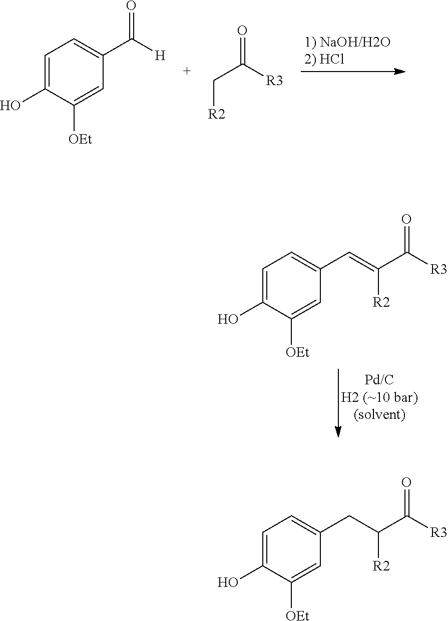

in which synthetic pathway (A):
   either R2 represents a hydrogen atom and R3 represents a linear (saturated) C1-C6 alkyl radical, optionally substituted with a hydroxyl group; or else a linear C2-C6 alkenyl (C═C unsaturated) radical, or alternatively a linear C2-C12 alkenyl radical substituted with a hydroxyl group;
   or R2 represents a methyl or ethyl radical, and R3 represents a linear (saturated) C1-C6 alkyl radical, optionally substituted with a hydroxyl group; or else a linear C2-C6 alkenyl (C═C unsaturated) radical, or alternatively a linear C2-C12 alkenyl radical substituted with a hydroxyl group.

In accordance with such a reaction scheme, the process for preparing the compound 4-(3-ethoxy-4-hydroxyphenyl)butan-2-one comprises at least the following successive steps: a) at least one step of reacting ethylvanillin with at least one carbonyl compound in the presence of sodium hydroxide and water to yield the compound of formula (W) below:

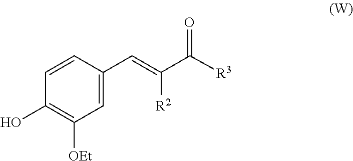

b) at least one step (b) of hydrogenation of the compound of formula (W), in the presence of at least one palladium (Pd) on carbon catalyst, at a pressure of approximately 10 bar, to yield ethylzingerone.

The process (A) for preparing compounds of formula (I), in particular the compound 4-(3-ethoxy-4-hydroxyphenyl)butan-2-one of formula (I″), does not lead to entirely satisfactory yields by conventional preparation methods (see for example WO 2011/039445, J. Asian Natural Products Research, 8(8), 683-688 (2006); Helv. Chimica Acta, 89(3), 483-495 (2006); Chem. Pharm. Bull., 54(3), 377-379 (2006); and Bioorg. Med. Chem. Lett., 14(5), 1287-1289 (2004)) and the implementation thereof needs to be further optimized, in particular in terms of the number of steps and/or the quality of the product on an industrial scale. In addition, the compounds of formula (I) and in particular the compound of formula (I″) have an odour specific thereto of eugenol (notably clove) spicy round vanilla type. However, such a preparation process (A) has the drawback of generating impurities, some of which have a pronounced odour, notably with a strong metallic and fusing character, which is very unpleasant and proves to be particularly difficult to eliminate. This unpleasant odour is due in particular to the presence of 2-ethoxy-4-n-butylphenol, the odour of which has these undesirable strong metallic fusing notes. In most cases, this disagreeable and persistent odour dominates the generally round vanilla odour of 4-(3-ethoxy-4-hydroxyphenyl)butan-2-one, which is incompatible with industrial scale development for applications, will in particular cosmetic applications. The same applies to other less odoriferous impurities such as 2-ethoxy-4-(3-hydroxybutyl)phenol, the amounts of which it is desired to minimize, or even eliminate, at the end of the preparation process.

In particular, reaction step (a) leads to a certain number of impurities, notably to dimers, in particular to a dimer of the compound of formula (W), a dimer of the carbonyl compound and mixed dimers resulting from the reaction between the compound of formula (W) and the carbonyl compound. In addition, once the reaction step (i) is completed, the reaction medium may still contain residual ethylvanillin which has not reacted with the carbonyl compound.

Such impurities, dimers or other impurities, may also be responsible for other impurities generated during the reaction step (b) (hydrogenation step) which are liable to have a pronounced and unpleasant odour.

Various purification operations, for example by washing, recrystallization and/or re-slurrying, carried out once or even several times in succession, were thus used in order to reduce or even eliminate the dimer impurities and other impurities and also the residual ethylvanillin from the reaction medium once the reaction step (a) was completed.

However, such purification operations, including those carried out several times in succession, did not significantly improve the degree of purity obtained on conclusion of reaction step (a).

More generally, such purification operations, carried out both on conclusion of reaction step (a) and of reaction step (b), did not make it possible to reduce or even eliminate the impurities generated during the process of preparing ethylzingerone and/or its derivatives, including impurities that give off an unpleasant and disagreeable, fusing and metallic eugenol odour, and have the drawback of reducing the yield.

As a result, the process for preparing 4-(3-ethoxy-4-hydroxyphenyl)butan-2-one and/or derivatives thereof, conventionally used in the prior art, does not lead to a degree of purity and yield that are sufficiently satisfactory for reproducible and robust industrial-scale production, in particular in the field of cosmetics.

In view of the above, there is therefore a real need to implement a novel process for preparing 4-(3-ethoxy-4-hydroxyphenyl)butan-2-one and/or compounds derived from 4-(3-ethoxy-4-hydroxyphenyl)butan-2-one, corresponding to formula (I), as described below, which does not have the abovementioned drawbacks, leading in particular to an improved degree of purity, a higher yield and the implementation of which is further optimized, specifically on an industrial level, in terms of the number of steps and/or the quality of the product on an industrial scale, without giving rise to the release of an unpleasant and unacceptable odour.

In particular, one of the objectives of the present invention is to provide a process for preparing compounds of formula (I), as described below, during which the impurities, including those having a pronounced and unpleasant odor, are capable of being minimized or even eliminated, in order to be able to be effectively implemented on an industrial scale, in particular for cosmetic applications.

In other words, one of the objectives of the present invention is to provide a process for preparing compounds of formula (I), the use of which does not lead to a release of bad odours, thus making it suitable for industrial applications, in particular cosmetic applications.

Therefore, the present invention relates in particular to a process for preparing at least one compound of formula (I) below:

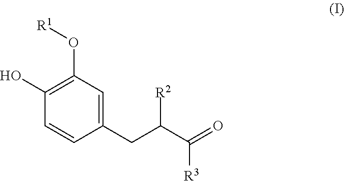

in which formula (I):
   R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
   R² represents a hydrogen atom or a C₂-C₄ alkyl group, such as methyl or ethyl; preferably R² represents a hydrogen atom;
   R³ represents a linear or branched, saturated or unsaturated hydrocarbon group comprising from 1 to 12 atoms, optionally substituted with an aryl group; the aryl group preferably being a phenyl group optionally substituted with one or more groups chosen from hydroxyl, C₁-C₄ alkyl group, C₁-C₄ alkoxy group and combinations thereof; preferably R³ represents a C₁-C₆ alkyl group; preferably a C₁-C₄ alkyl group;
and also one of the optical isomers or geometrical isomers thereof, and/or one of the salts thereof with an organic or inorganic acid or base, and/or one of the solvates thereof, such as the hydrates;
said process taking place according to the following synthesis scheme (1)

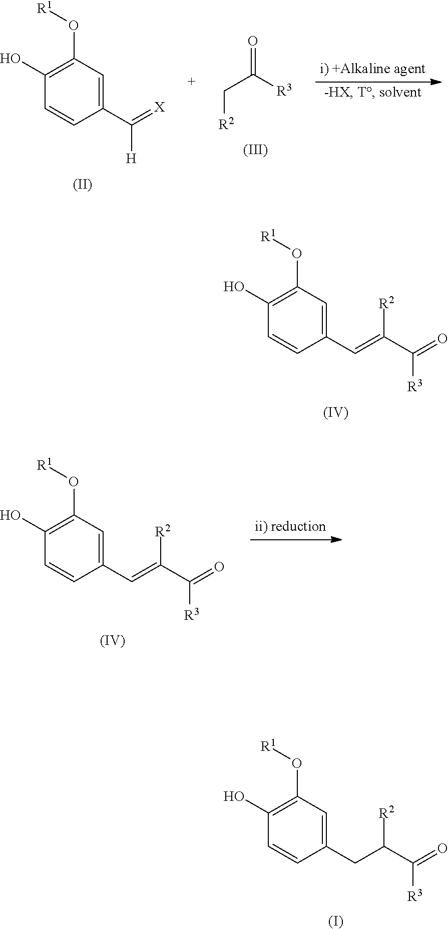

in which synthetic pathway (1):
   X represents a heteroatom chosen from oxygen and sulfur, preferably an oxygen atom,
   R¹, R² and R³ have the same meanings as in the formula (I);
said process being characterised in that it comprises:
   at least one step (i) of reacting the compound of formula (II) in the presence of:
      at least one compound of formula (III),
      at least one (non)polar (a)protic solvent, and
      at least one alkaline agent in an excess molar amount relative to the compound of formula (II),
   in order to result in the formation of at least one precipitate including at least one compound of formula (IV), geometrical isomers thereof and/or one of the one of the salts thereof with an organic or inorganic base; and
   at least one step (i1) of separating the precipitate from the reaction medium of step (i), then
   at least one step (i2) of acidifying the precipitate using at least one inorganic or organic, preferably inorganic acidifying agent, before carrying out the reaction step (ii).

In other words, once the precipitate, including at least said compound of formula (IV), the geometrical isomers thereof and/or one of the salts thereof with an organic or inorganic, preferably inorganic base, is formed on conclusion of step (i), said precipitate is then isolated from the reaction medium and then acidified, using at least one inorganic or organic acidifying agent, before carrying out reaction step (ii).

After the separation step (i1) and the acidification step (i2), the compound of formula (IV) is used in a reduction step (ii) to yield the compound of formula (I).

The process according to the invention thus makes it possible to achieve the objectives as described above, that is to say that it results in a compound of formula (I) having a high degree of purity with a satisfactory yield while exhibiting an optimized implementation on the industrial level, notably which is robust and reproducible, in particular with respect to the processes conventionally used in the prior art.

In particular, the process according to the invention has the advantage of minimizing or even eliminating impurities, including those usually having a pronounced and unpleasant odour, in particular, impurities which give off an unpleasant odour with fusing and metallic notes or smoky and woody notes that are particularly difficult to remove in the processes conventionally used in the prior art.

The process according to the invention thus has the advantage of minimizing, or even eliminating, the unpleasant and disagreeable odours (in particular with fusing and metallic notes) occurring in the processes conventionally used in the prior art.

The process according to the invention thus makes it possible to result in a compound of formula (I) having a high purity (greater than or equal to 95% by HPLC).

Advantageously, the separation of the precipitate from the reaction medium on conclusion of step (i) makes it possible to envisage recycling a portion of the alkaline agent used in excess in order to improve the performance of step (i).

The present invention also relates to a compound corresponding to formula (I′) below:

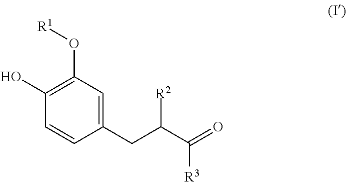

in which formula (I′):
   R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
   R² represents a hydrogen atom or a C₂-C₄ alkyl group, such as methyl or ethyl;
   R³ represents a branched, saturated or unsaturated hydrocarbon group comprising from 1 to 12 carbon atoms, preferably R³ represents a branched, saturated or unsaturated hydrocarbon group comprising from 3 to 12 carbon atoms; more preferably R³ represents a branched C₁-C₆ alkyl group, more preferentially R³ represents a branched C₃-C₆ alkyl group; especially R³ represents a branched C₃-C₄ alkyl group, in particular a branched C₄ alkyl group;
and also one of the optical isomers or geometrical isomers thereof, and/or one of the salts thereof with an organic or inorganic acid or base, and/or one of the solvates thereof, such as the hydrates.

Furthermore, the present invention also relates to a composition comprising, in a physiologically acceptable medium, at least one compound of formula (I′), the salts, and/or isomers and/or solvates thereof, as described above.

Other subject-matters, characteristics, aspects and advantages of the invention will become still more clearly apparent on reading the description and the example which follow.

In that which follows and unless otherwise indicated, the limits of a range of values are included within this range, in particular in the expressions “of between” and “ranging from . . . to . . . ”.

Moreover, the expression “at least one” used in the present description is equivalent to the expression “one or more”.

In addition, the expression “at least” used in the present description is equivalent to the expression “greater than or equal to”. Finally, in a way known per se, the term “Cₙ” compound or group denotes a compound or a group containing, in its chemical structure, “n” carbon atoms.

The HPLC purity corresponds to the relative purity expressed as percentage of area, measured at the maximum absorption wavelength (Amax) of the product analysed.

Within the meaning of the present invention, the terms “alkaline agent” and “basifying agent” are used without distinction.

The basifying agent(s) can be inorganic alkaline agents, preferably chosen from the group constituted of alkali metal hydroxides or alkaline earth metal hydroxides, such as lithium hydroxide, sodium hydroxide, potassium hydroxide, alkali metal or alkaline earth metal (bi)carbonates, such as sodium or potassium (bi)carbonates, and mixtures thereof. The basifying agent(s) can be organic alkaline agents, preferably chosen from the group constituted of mono(C₁-C₆)(hydroxy)alkylamines, di(C₁-C₆)(hydroxy)alkylamines, tri(C₁-C₆)(hydroxy)alkylamines (preferably tri(C₁-C₆)(hydroxy)alkylamines), saturated or unsaturated cyclic amines which are aromatic, such as pyridine, or non-aromatic, optionally substituted by one or more (C₁-C₄)alkyl groups, such as tetrahydropyridine optionally substituted by one or more (C₁-C₄)alkyl groups, piperidine optionally substituted by one or more (C₁-C₄)alkyl groups or piperazine optionally substituted by one or more (C₁-C₄)alkyl groups. Preferably, the alkaline agents of the invention are tertiary amines.

Preferably, the basifying agents are chosen from the group constituted of alkali metal or alkaline earth metal hydroxides, in particular sodium hydroxide, alkali metal or alkaline earth metal (bi)carbonates, in particular sodium or potassium (bi)carbonates, and tri(C₁-C₆)(hydroxy)alkylamines, in particular tri(C₁-C₆)alkylamines, notably triethylamine.

More preferentially, the basifying agents are inorganic and are chosen from the group constituted of alkali metal or alkaline earth metal hydroxides, alkali metal or alkaline earth metal (bi)carbonates, and mixtures thereof, in particular alkali metal or alkaline earth metal hydroxides, in particular sodium hydroxide.

### Preparation Process

As indicated above, the process according to the invention takes place according to the reaction scheme (1), as described above, and comprises:
at least one step (i) comprising at least the reaction of at least one compound of formula (II) in the presence of at least one compound of formula (III), at least one (non)polar (a)protic solvent and at least one alkaline agent, present in an excess molar amount relative to the compound of formula (II), to lead to the formation of a precipitate including at least the compound of formula (IV), the geometrical isomers thereof and/or one of the salts thereof with an organic or inorganic base.
at least one step (i1) of separating the precipitate from the reaction medium of step (i), then
at least one step (i2) of acidifying the precipitate using at least one inorganic or organic, preferably inorganic acidifying agent, before carrying out the reaction step (ii).

According to the present invention, step (i) comprises the reaction of:
at least one compound of formula (II), in which R¹ and X have the same meanings as those indicated above,
   preferably, in formula (II), X represents an oxygen atom, in the presence of:
at least one compound of formula (III), in which R² and R³ have the same meanings as those indicated above,
   preferably, in formula (III), R² represents a hydrogen atom and R³ represents a C₁-C₆ alkyl group, preferably a C₁-C₄ alkyl group, preferably a methyl or ethyl group, in particular a methyl group;
   advantageously, said compound of formula (III) is present in the reaction medium of step (i) in an amount less than or equal to 5 molar equivalents, which makes it possible to improve the kinetics of reaction step (i), more preferentially in an amount ranging from 2 to 5 molar equivalents, better still in an amount ranging from 2.5 to 5 molar equivalents, in particular in an amount of 4.5 molar equivalents, relative to the compound of formula (II);
at least one (non)polar (a)protic solvent, preferably at least one polar protic solvent, such as water,
   preferably, as a mixture with at least one (non)polar (a)protic organic solvent, preferably (non)polar aprotic organic solvent, such as nonpolar aprotic organic solvents, preferably having a low dielectric constant and dipole moment, preferably having a dielectric constant E ranging from 1 to 11 and a dipole moment p ranging from 0 to 2, in particular those chosen from the group constituted of n-hexane, cyclohexane, 1,4-dioxane, carbon tetrachloride (CCl₄), benzene, tetrachloroethylene (Cl₂C═Cl₂C), toluene, carbon disulfide (CS₂), trichloroethylene (Cl₂C═CHCl), diethyl ether (Et₂O), ethyl acetate (CH₃C(O)OEt or AcOEt), dimethyl ether (DME), tetrahydrofuran (THF), methyltetrahydrofuran (2-MeTHF), dichloromethane (CH₂Cl₂), dichloroethane (C₁CH₂CH₂C₁) and mixtures thereof;
at least one organic or inorganic, preferably inorganic alkaline agent, omore preferentially chosen from the group constituted of alkali metal or alkaline earth metal hydroxides, alkali metal or alkaline earth metal (bi)carbonates, and mixtures thereof, in particular alkali metal or alkaline earth metal hydroxides, in particular sodium hydroxide;
said alkaline agent being in an excess molar amount relative to the compound of formula (II),
   preferably, the alkaline agent is in an amount greater than or equal to 2 molar equivalents, more preferentially ranging from 2 to 5 molar equivalents, for example 4.5 molar equivalents, relative to the compound of formula (II);
in order to result in the formation of at least one precipitate including at least the compound of formula (IV), the geometrical isomers thereof and/or one of the one of the salts thereof with an organic or inorganic base.

Preferably, the alkaline agent(s) of step (i) is or are inorganic alkaline agents chosen from the group constituted of alkali metal or alkaline earth metal hydroxides, alkali metal or alkaline earth metal (bi)carbonates, and mixtures thereof, in particular alkali metal or alkaline earth metal hydroxides, in particular sodium hydroxide.

Preferably, the alkaline agent(s) of step (i) is or are inorganic alkaline agents chosen from the group constituted of alkali metal or alkaline earth metal hydroxides, in particular sodium hydroxide.

Preferably, the alkaline agent(s) of step (i) is or are inorganic alkaline agents selected from the group constituted of alkali metal or alkaline earth metal hydroxides, in particular sodium hydroxide and present in an amount greater than or equal to 2 molar equivalents, more preferentially in an amount ranging from 2 to 5 molar equivalents, relative to the compound of formula (II).

Preferably, step (i) is carried out at a temperature below or equal to 40° C., preferably at a temperature ranging from 20° C. to 35° C., more preferentially at a temperature ranging from 25° C. to 35° C., in particular at a temperature of 30° C. D 1° C., which makes it possible to improve both the kinetics and the quality of the reaction of step (i), in particular the quality of the compound of formula (IV).

Advantageously, step (i) is carried out at a temperature below or equal to 40° C., preferably at a temperature ranging from 20° C. to 35° C., more preferentially at a temperature ranging from 25° C. to 35° C., in particular at a temperature of 30° C.+/−1° C., and said alkaline agent is present in an amount ranging from 2 to 5 molar equivalents relative to the compound of formula (II) which also makes it possible to improve both the kinetics and the quality of the reaction of step (i).

Advantageously, step (i) is carried out at a temperature below or equal to 40° C., preferably at a temperature ranging from 20° C. to 35° C., more preferentially at a temperature ranging from 25° C. to 35° C., in particular at a temperature of 30° C.+/−1° C., for a period ranging from 30 minutes to 24 hours, preferably ranging from 6 hours to 18 hours, more preferentially ranging from 10 hours to 15 hours, and in particular is 12 hours.

Advantageously, step (i) is carried out with:
at least one alkaline agent, as defined above, in an amount ranging from 2 to 5 molar equivalents relative to the compound of formula (II),
at a reaction temperature below or equal to 40° C., preferably at a temperature ranging from 20° C. to 35° C., more preferentially at a temperature ranging from 25° C. to 35° C., notably at a temperature of 30° C.+/−1° C.,
for a period ranging from 30 minutes to 24 hours, preferably ranging from 6 hours to 18 hours, more preferentially ranging from 10 hours to 15 hours, notably is 12 hours.

Preferably, step (i) is carried out with at least one compound of formula (II) in the presence of:
at least one compound of formula (III) present in an amount ranging from 2 to 5 molar equivalents,
   more preferentially in an amount ranging from 2.5 to 5 molar equivalents, in particular in an amount of 4.5 molar equivalents, relative to the compound of formula (II);
at least one (non)polar (a)protic solvent, preferably at least one polar protic solvent, such as water,
   optionally as a mixture with at least one nonpolar aprotic organic solvent,
at least one inorganic alkaline agent, preferably selected from the group constituted of alkali metal or alkaline earth metal hydroxides, alkali metal or alkaline earth metal (bi)carbonates, and mixtures thereof, in an amount ranging from 2 to 5 molar equivalents, in particular in an amount of 4.5 molar equivalents, relative to the compound of formula (II);
at a temperature below or equal to 40° C., preferably at a temperature ranging from 20° C. to 35° C., more preferentially at a temperature ranging from 25° C. to 35° C., notably at a temperature of 30° C.+/−1° C.

Advantageously, step (i) is carried out with at least one compound of formula (II) in the presence of:
at least one compound of formula (III) present in an amount of 4.5 molar equivalents, relative to the compound of formula (II),
at least one polar protic solvent, such as water, optionally as a mixture with at least one nonpolar aprotic organic solvent,
at least one inorganic alkaline agent selected from the group constituted of alkali metal or alkaline earth metal hydroxides, in an amount ranging from 2 to 5 molar equivalents, relative to the compound of formula (II);
at a reaction temperature ranging from 20° C. to 35° C., more preferentially at a temperature ranging from 25° C. to 35° C., notably at a temperature of 30° C.+/−1° C.,
the reaction time varying from 6 hours to 18 hours, preferably from 10 hours to 15 hours, and notably is 12 hours.

Particularly, the reaction step (i) of the process according to the invention is carried out at atmospheric pressure (1.013×10⁵ Pa).

Preferably, the reaction step (i) of the process according to the invention is carried out with mechanical stirring.

Preferably, the reaction step (i) of the process according to the invention is carried out in an industrial, particularly metallic, reactor.

Preferably, the reaction step (i) of the process according to the invention is carried out:
either by adding said alkaline agent, as defined above, preferably an aqueous solution comprising at least said alkaline agent, to a reaction medium comprising at least one compound of formula (II), at least one compound of formula (III), and at least one (a)polar (a)protic solvent, preferably a polar protic solvent, such as water, preferably to at least one aqueous solution comprising at least one compound of formula (II) and at least one compound of formula (III),
or by adding at least one compound of formula (II), at least one compound of formula (III) and at least one (a)polar (a)protic solvent, preferably at least one polar protic solvent, such as water, to a reaction medium comprising at least said alkaline agent and at least one polar protic solvent, such as water,
or by simultaneously adding at least one compound of formula (II) and a solution comprising at least one polar protic solvent, such as water, and at least one alkaline agent to a reaction medium comprising at least one compound of formula (III), at least one polar protic solvent, such as water, and at least said alkaline agent; preferably the alkaline agent is identical.

Advantageously, the reaction step (i) of the process according to the invention is carried out by adding said alkaline agent, as defined above, preferably an aqueous solution comprising at least said alkaline agent, to a reaction medium comprising at least one compound of formula (II), at least one compound of formula (III), and at least one (a)polar (a)protic solvent, preferably a polar protic solvent, such as water, preferably to at least one aqueous solution comprising at least one compound of formula (II) and at least one compound of formula (III).

In accordance with this advantageous embodiment, the chemical degradation of the compound of formula (II) and/or of the compound of formula (III) by said alkaline agent is thus minimized over time.

In particular, such a chemical degradation of the compound of formula (II) and/or of the compound of formula (III) by said alkaline agent can be observed owing to the appearance of a brownish colour over time which can be monitored by chromatography, preferably by HPLC chromatography, in particular with a UV detector.

As a variant, the reaction step (i) of the process according to the invention can be carried out by simultaneously adding at least one compound of formula (II) and a solution comprising at least one polar protic solvent, such as water, and at least one alkaline agent to a reaction medium comprising at least one compound of formula (III), at least one polar protic solvent, such as water, and at least said alkaline agent; preferably the alkaline agent is identical.

Preferably, during step (i), the addition of said alkaline agent(s) is carried out over a period ranging from 10 minutes to 3 hours, more particularly over a period ranging from 30 minutes to 1 hour 30 minutes, in particular over one hour, preferably at a temperature below or equal to 40° C., preferably at a temperature ranging from 25° C. to 32° C., in particular at a temperature of 25° C.

In other words, preferably, the addition time of said alkaline agent(s) varies from 10 minutes to 3 hours, more particularly over a period ranging from 30 minutes to 1 hour 30 minutes, in particular over one hour, preferably at an addition temperature below or equal to 40° C., preferably at an addition temperature ranging from 25° C. to 32° C., in particular at an addition temperature of 25° C.

Preferably, once the alkaline agent(s) has(have) been added to the reaction medium of step (i); said reaction medium is maintained at a temperature below or equal to 40° C., preferably at a temperature ranging from 20° C. to 35° C., more preferentially at a temperature ranging from 25° C. to 35° C., in particular at a temperature of 30° C.

Preferably, the progress of the reaction (i), in particular of the formation of the compound of formula (IV) and also the geometrical isomers thereof, the salts thereof with an organic or inorganic base, preferably an inorganic base is monitored by chromatography or by GC-MS, preferably by HPLC chromatography, in particular with a UV detector.

As indicated above, the process according to the invention comprises:
at least one step (i), as described above, resulting in the formation of at least one precipitate including at least the compound of formula (IV), geometrical isomers thereof and/or one of the one of the salts thereof with an organic or inorganic base, preferably an inorganic base,
at least one step (i1) of separating the precipitate from the reaction medium of step (i), preferably by filtration, more preferentially by pressure filtration, for example using a piston, then
at least one step (i2) of acidifying the precipitate using at least one inorganic or organic, preferably inorganic acidifying agent, before carrying out the reaction step (ii).

Preferably, the separation step (i1) is carried out by filtration, more preferentially by pressure filtration, by centrifugal filtration, or is carried out by centrifuging.

Preferably, the acidifying agent(s) of step (i2) is or are inorganic acidifying agents, in particular of H⁺Hal⁻ type, with Hal representing a halogen atom chosen from the group constituted of chlorine, bromine and iodine, more preferentially, the acidifying agent is hydrochloric acid.

Preferably, once the reaction step (i) is completed, the reaction medium is cooled to a temperature below or equal to 25° C., preferably in a temperature range of from 1° C. to 12° C., preferably ranging from 3° C. to 8° C., and then the precipitate is separated from the reaction medium, preferably by filtration, more preferentially by pressure filtration.

In other words, once reaction step (i) is completed, the reaction medium is preferably cooled to a temperature below or equal to 25° C., preferably ranging from 1° C. to 12° C., preferably ranging from 3° C. to 8° C., before the separation step (i1) is carried out.

Once separated from the reaction medium, the precipitate may be washed one or more times with at least one aprotic organic solvent, for example acetone, and then may be washed one or more times with at least one protic polar solvent, such as water.

Preferably, the acidification step (i2) comprises:
in particular, at least the introduction of the precipitate, which may be in powder form, into a reactor,
   preferably with at least one polar protic solvent, such as water, and optionally at least one (non)polar aprotic organic solvent, such as acetone, in particular in an amount ranging from 10% of polar protic solvent and 90% of (non)polar aprotic solvent (by volume) to 90% of polar protic solvent and 10% of (non)polar aprotic solvent (by volume), notably 50% of polar protic solvent and 50% of (non)polar aprotic solvent (by volume),
at least the addition of at least one inorganic or organic, preferably inorganic, acidifying agent, to the reaction medium comprising said precipitate, preferably until a homogeneous suspension in the solvent(s) as defined above is obtained;
   preferably, the acidifying agent is inorganic, in particular hydrochloric acid,
   preferably, the pH of the reaction medium of step (i2) is less than 7, more preferentially varies from 1 to 6, even more preferentially from 2 to 5, better still less than 4,
   preferably, the temperature of the reaction medium of step (i2) is maintained at a temperature below or equal to 25° C., more preferentially in a temperature range of from 1° C. to 20° C., better still from 3° C. to 20° C.,
   more preferentially, during the addition of said acidifying agent, the reaction medium is maintained at a temperature below or equal to 25° C., notably ranging from 5° C. to 25° C., in particular from 15° C. to 20° C.;
   more preferentially, after the addition of said acidifying agent, the reaction medium is maintained at a temperature below or equal to 15° C., notably ranging from 1° C. to 15° C., in particular from 2° C. to 10° C., more particularly ranging from 3° C. to 8° C.

Once step (i2) has been carried out, the precipitate is preferably filtered off and then optionally washed one or more times with at least one polar protic solvent, such as water, preferably, the precipitate is washed twice with at least one polar protic solvent, such as water.

After steps (i1) and (i2) have been carried out, the compound of formula (IV) advantageously has a high purity, in particular greater than or equal to 97% measured by HPLC.

After steps (i1) and (i2) have been carried out, the compound of formula (IV) is preferably dissolved in at least one (non)polar aprotic organic solvent, preferably in at least one nonpolar aprotic organic solvent, optionally as a mixture with at least one polar protic solvent, such as water, before step (ii) is carried out.

Preferably, the nonpolar aprotic organic solvent has a dielectric constant E ranging from 1 to 11 and a dipole moment p ranging from 0 to 2, and may be chosen from the group constituted of n-hexane, cyclohexane, 1,4-dioxane, carbon tetrachloride (CCl₄), benzene, tetrachloroethylene (Cl₂C═Cl₂C), toluene, carbon disulfide (CS₂), trichloroethylene (Cl₂C═CHCl), diethyl ether (Et₂O), ethyl acetate (CH₃C(O)OEt or AcOEt), dimethyl ether (DME), tetrahydrofuran (THF), methyltetrahydrofuran (2-MeTHF), dichloromethane (CH₂Cl₂), dichloroethane (C₁CH₂CH₂C₁) and mixtures thereof.

The pH of the reaction medium, when dissolving the compound of formula (IV) with at least one (non)polar aprotic organic solvent, is preferably greater than 5.

The compound of formula (IV) can be dissolved in at least one (non)polar aprotic organic solvent, preferably in at least one nonpolar aprotic organic solvent, optionally as a mixture with at least one polar protic solvent, such as water, in the presence of at least one alkaline agent having a pKa varying from 8 to 11, preferably from 9 to 11, in particular chosen from the group constituted of alkali metal or alkaline earth metal (bi)carbonates.

Preferably, when dissolving the compound of formula (IV) with at least one (non)polar aprotic organic solvent:
the reaction medium is heated to a temperature above or equal to 40° C., in particular to a temperature of 45° C., and then
the reaction medium is cooled to a temperature below 25° C., in particular to a temperature of 20° C., and then
the reaction medium is left to settle in order to then remove the aqueous phase,
the resulting organic phase is then washed, one or more times, with water, preferably at a temperature ranging from 15° C. to 25° C., in particular at a temperature of 20° C., and then is left to settle,
the water is then removed, preferably by distillation using Dean-Stark apparatus, in particular at a temperature above or equal to 80° C., in particular at a temperature ranging from 80° C. to 90° C., preferably from 82° C. to 85° C., according to another variant, the water can be removed using Dean-Stark apparatus under vacuum at a temperature below 80° C.

Advantageously, the process according to the invention comprises:
at least one step (i), as described above, resulting in the formation of at least one precipitate including at least the compound of formula (IV), geometrical isomers thereof and/or one of the one of the salts thereof with an organic or inorganic base, preferably an inorganic base,
at least one step (i1) of separating the precipitate from the reaction medium of step (i), preferably by filtration, more preferentially by pressure filtration, for example using a piston, then
at least one step (i2) of acidifying the precipitate using at least one inorganic or organic, preferably inorganic acidifying agent, before carrying out the reaction step (ii), and then
at least one step of dissolving the compound of formula (IV) in at least one (non)polar aprotic organic solvent, preferably in at least one apolar aprotic organic solvent, as defined above, before carrying out step (ii).

As indicated above, the process according to the invention comprises a step (ii) of reducing the compound of formula (IV) to yield at least one compound of formula (I).

Step (ii) of reducing the compound of formula (IV) can be carried out in the presence or in the absence of at least one solvent.

Preferably, the reduction step (ii) can be a catalytic reduction, preferably a reduction by catalytic hydrogenation, or a reduction by conventional reducing agents known to those skilled in the art, such as alkali metal hydrides, for instance alkali metal borohydrides such as NaBH₄, and alkali metal aluminum tetrahydrides such as LiAlH₄.

Preferably, the reduction step (ii) is a reduction by catalytic hydrogenation.

Advantageously, the compound of formula (IV) is dissolved in at least one (non)polar aprotic organic solvent, preferably in at least one nonpolar aprotic organic solvent, as defined above, and is then hydrogenated to yield at least one compound of formula (I).

Preferably, step (ii) is a hydrogenation carried out in the presence of at least one catalyst.

The catalyst is preferably palladium (Pd), for example palladium on charcoal (Pd/C), palladium on alumina (Pd/Al), palladium on AlSi or palladium on barium sulfate (BaSO₄), ruthenium (Ru) or nickel (Ni), more particularly chosen from Pd and Ru.

Preferably, the catalyst is palladium (Pd), for example on charcoal (Pd/C).

Preferably, the catalyst is present in the reaction medium of step (ii) in an amount ranging from 0.05% to 10%, preferably in an amount ranging from 0.1% to 10% by weight, preferably in a content ranging from 0.5% to 1% relative to the total weight of the reaction medium of step (ii).

Preferably, step (ii) is a hydrogenation carried out in the presence of at least one catalyst, preferably palladium (Pd), for example on carbon (Pd/C), in particular in the presence of at least one alkaline agent having a pKa ranging from 8 to 11, preferably from 9 to 11, notably from 9 to 10, preferably chosen from the group constituted of alkali metal or alkaline earth metal (bi)carbonates and mixtures thereof.

As a variant, said alkaline agent having a pKa ranging from 8 to 11, preferably from 9 to 11, notably from 9 to 10, may be introduced into the reaction medium comprising at least the compound of formula (IV) prior to carrying out the reduction step (ii), in particular when the compound of formula (IV) is dissolved in at least one (non)polar aprotic organic solvent as described above.

Preferably, the reduction step (ii) is carried out in a reaction medium, the pH of which varies from 8 to 11, preferably from 9 to 10, which makes it possible to further limit the formation of impurities.

Preferably, the reduction step (ii) is carried out at a temperature greater than or equal to 20° C., more preferably greater than or equal to 50° C., still more preferably greater than or equal to 80° C. According to one preferred embodiment, the temperature is in a range between 20° C. and 150° C., more particularly in a range between 30° C. and 100° C., preferably in a range between 50° C. and 80° C.

On conclusion of reaction step (ii), the reaction medium is preferably cooled to a temperature below or equal to 45° C. before optionally filtering off the catalyst.

The reaction medium may be rinsed with at least one nonpolar aprotic organic solvent, such as methyl-THF.

Preferably, on conclusion of reaction step (ii), the reaction medium comprising the compound of formula (I) can be washed by one or more acid-base washing operations, in particular by the successive addition of at least one organic or inorganic, preferably inorganic, alkaline agent, and at least one organic or inorganic, preferably inorganic, acidifying agent.

By way of example, preferably:
at least one alkaline agent, preferably selected from the group constituted of alkali metal or alkaline earth metal hydroxides, in particular sodium hydroxide, and at least one polar protic solvent, such as water, are added to the reaction medium comprising the compound of formula (I),
the reaction medium is left to settle and then the aqueous phase is removed from the reaction medium,
the resulting organic phase is then washed one or more times with water which is then removed,
at least one, preferably inorganic, acidifying agent and water are added to the resulting organic phase; the resulting reaction medium has in particular a pH of less than 2,
the aqueous phase is then removed,
the resulting organic phase is washed one or more times with water and then the nonpolar aprotic organic solvent, possibly present in the organic phase, is removed by vacuum distillation, in particular at a temperature ranging from 75° C. to 85° C.

On conclusion of step (ii), preferably after one or more acid-base washing steps, the compound of formula (I) is advantageously purified by distillation, preferably short-path distillation, in order to remove any remaining volatile or nonvolatile impurities.

The compound of formula (I) can then be isolated with a yield of at least 90% and with a purity of at least 95%.

As indicated above, the process according to the invention is a process for preparing at least one compound of formula (I) as defined above, one of the optical isomers or geometrical isomers thereof, and/or one of the salts thereof with an organic or inorganic acid or base, and/or one of the solvate thereof such as hydrates.

The salts of the compounds of formula (I) comprise the conventional non-toxic salts of said compounds, such as those formed from an acid or base.

Mention may in particular be made, as salts of the compounds of formula (I), of:
the salts obtained by addition of the compound of formula (I) with an inorganic base, such as sodium hydroxide, potassium hydroxide, calcium hydroxide, ammonium hydroxide, magnesium hydroxide, lithium hydroxide, and sodium, potassium or calcium carbonates or hydrogen carbonates, for example;
the salts obtained by addition of the compound of formula (I) with an organic base, such as a primary, secondary or tertiary alkylamine, for example triethylamine or butylamine. This primary, secondary or tertiary alkylamine can comprise one or more nitrogen and/or oxygen atoms and can thus comprise, for example, one or more alcohol functions; mention may in particular be made of 2-amino-2-methylpropanol, ethanolamine, triethanolamine, 2-(dimethylamino)propanol, 2-amino-2-hydroxymethyl-1,3-propanediol or 3-(dimethylamino)propylamine.

Mention may also be made of the salts of amino acids, such as, for example, lysine, arginine, guanidine, glutamic acid or aspartic acid.

Advantageously, the salts of the compounds of formula (I) can be chosen from alkali metal or alkaline earth metal salts, such as sodium, potassium, calcium or magnesium salts, or ammonium salts.

The acceptable solvates of the compounds of formula (I) comprise conventional solvates, such as those formed during the preparation of said compounds as a result of the presence of solvents.

Examples that may be mentioned include solvates due to the presence of water or of linear or branched alcohols, such as ethanol or isopropanol.

The optical isomers are in particular enantiomers and diastereoisomers.

Preferably, in formula (I), R² represents a hydrogen atom.

Preferably, in formula (I), R¹ represents a linear or branched, preferably linear, C₁-C₄ alkyl group, more preferentially a linear, C₂-C₄ alkyl group, such as ethyl.

Preferably, in formula (I), R³ represents a linear or branched C₁-C₆ alkyl group, optionally substituted with an aryl group as described above.

Preferably, in formula (I), R² represents a hydrogen atom and/or R¹ represents a linear or branched, preferably linear C₁-C₄ alkyl group, more preferentially a linear C₂-C₄ alkyl group, such as ethyl and/or R³ represents a linear or branched C₁-C₆ alkyl group.

More preferentially, the process according to the invention is a process for preparing at least one compound of formula (T):

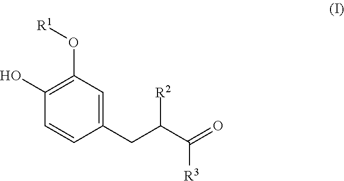

in which formula (I):
   R¹ represents a linear or branched, preferably linear, C₁-C₄ alkyl group, more preferentially a linear C₂-C₄ alkyl group, such as ethyl;
   R² represents a hydrogen atom;
   R³ represents a linear or branched, saturated or unsaturated hydrocarbon group comprising from 1 to 12 atoms, optionally substituted with an aryl group; the aryl group preferably being a phenyl group optionally substituted with one or more groups chosen from hydroxyl, C₁-C₄ alkyl group, C₁-C₄ alkoxy group and combinations thereof; preferably R³ represents a linear or branched C₁-C₆ alkyl group; preferably a linear or branched C₁-C₄ alkyl group, such as methyl or ethyl;

Preferably, in formula (I), R¹ represents a linear or branched C₂-C₄ alkyl group, in particular ethyl, R² represents a hydrogen atom, R³ represents a C₁-C₆ alkyl group; preferably a C₁-C₄ alkyl group, in particular methyl.

In other words, even more preferentially, the process according to the invention is a process for preparing at least one compound of formula (I):

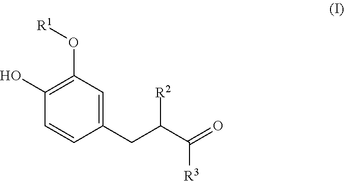

in which formula (I):
   R¹ represents a linear or branched C₂-C₄ alkyl group, in particular ethyl;
   R² represents a hydrogen atom;
   R³ represents a linear or branched C₁-C₆ alkyl group; preferably a C₁-C₄ alkyl group, in particular methyl.

Advantageously, the process according to the invention makes it possible to prepare one or more compounds of formula (I) chosen from the group constituted of the compounds corresponding to_formula (I″) below:

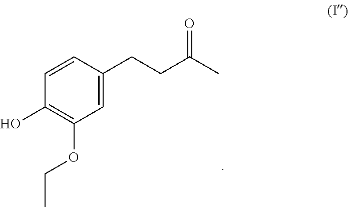

and also the optical isomers, geometrical isomers, and tautomers thereof, and also the salts thereof with an organic or inorganic acid or base, and the solvates thereof, such as the hydrates.

Preferably, the process according to the invention makes it possible to prepare one or more compounds of formula (I) chosen from the group constituted of the compounds corresponding to formula (I′) below:

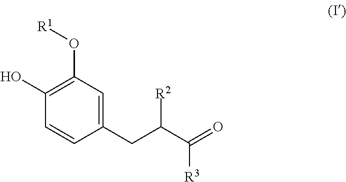

in which formula (I′):
   R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
   R² represents a hydrogen atom or a C₂-C₄ alkyl group, such as methyl or ethyl;
   R³ represents a branched, saturated or unsaturated hydrocarbon group comprising from 1 to 12 carbon atoms, preferably R³ represents a branched, saturated or unsaturated hydrocarbon group comprising from 3 to 12 carbon atoms; more preferably R³ represents a branched C₁-C₆ alkyl group, more preferentially R³ represents a branched C₃-C₆ alkyl group; especially R³ represents a branched C₃-C₄ alkyl group, in particular a branched C₄ alkyl group;
and also one of the optical isomers or geometrical isomers thereof, and/or one of the salts thereof with an organic or inorganic acid or base, and/or one of the solvates thereof, such as the hydrates.

The process according to the invention thus makes it possible to prepare the compound(s) of formula (I′) preferentially according to the following synthesis scheme (1″):

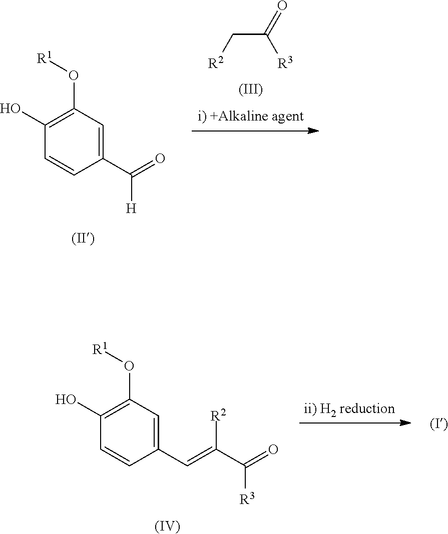

in which synthetic pathway (1″):
   R¹, R² and R³ have the same meanings as those indicated in formula (I′);

More preferentially, the process makes it possible to prepare the compound(s) of formula (I″) from ethylvanillin (II′a) according to the following synthesis scheme (1″′):

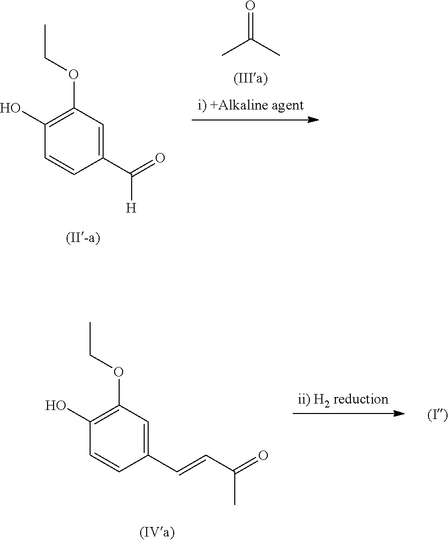

The process according to the invention for preparing the compound(s) of formula (I′) or (I″) comprises at least one step (i), at least one separation step (i1) and at least one acidification step (i2), as defined above.

The process according to the invention for preparing the compound(s) of formula (I′) or (I″) comprises at least one reduction step (ii) as described above.

Preferably, the process according to the invention for preparing the compound(s) of formula (I′) or (I″) comprises a step of dissolving the compound of formula (IV) or (TV′a), as described above.

Preferably, the process according to the invention for preparing the compound(s) of formula (I′) or (I″) comprises, in conclusion of step (ii), one or more acid-base washing steps and/or at least one purification step, preferably short-path distillation, in order to remove any remaining volatile or nonvolatile impurities.

### Compound of Formula (I′)

As indicated above, the present invention also relates to a compound of formula (I′) below:

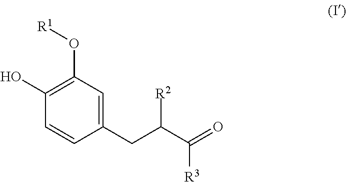

in which formula (I′):
   R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
   R² represents a hydrogen atom or a C₂-C₄ alkyl group, such as methyl or ethyl; preferably R² represents a hydrogen atom;
   R³ represents a branched, saturated or unsaturated hydrocarbon group comprising from 1 to 12 atoms; preferably R³ represents a branched, saturated or unsaturated hydrocarbon group comprising from 3 to 12 carbon atoms; more preferably R³ represents a branched C₁-C₆ alkyl group, more preferentially R³ represents a branched C₃-C₆ alkyl group; especially R³ represents a branched C₃-C₄ alkyl group, in particular a branched C₄ alkyl group;
and also one of the optical isomers or geometrical isomers thereof, and/or one of the salts thereof with an organic or inorganic acid or base, and/or one of the solvates thereof, such as the hydrates.

Preferably, in formula (I′), R³ represents a branched C₁-C₆ alkyl group, more preferentially R³ represents a branched C₁-C₄ alkyl group, in particular a branched C₄. alkyl radical.

Preferably, in formula (I′), R³ represents a branched C₁-C₆ alkyl group, more preferentially R³ represents a branched C₃-C₆ alkyl group, especially a branched C₁-C₄ alkyl group, even more preferably R³ represents a branched C₃-C₄ alkyl group, in particular a branched C₄ alkyl group.

According to a preferred embodiment, in formula (I′), R³ represents a branched C₃-C₆ alkyl group; especially R³ represents a branched C₃-C₄ alkyl group, in particular a branched C₄ alkyl group.

Preferably, in formula (I′), R² represents a hydrogen atom.

Preferably, in formula (I′), R¹ represents a C₁-C₄ alkyl group, more preferentially a C₂-C₄ alkyl group, such as ethyl.

Furthermore, the present invention also relates to a composition comprising at least one compound of formula (I′), the salts, and/or isomers and/or solvates thereof, as described above.

Preferably, the composition comprises at least one compound of formula (I′), the salts, and/or isomers and/or solvates thereof, as described above, in a content of greater than 10% by weight, relative to the total weight of the composition.

### Compound of Formula (I)

Another subject of the present invention is a compound of formula (I) or (I′), preferably (I″), and/or one of the salts thereof, and/or one of the geometrical or optical isomers thereof and/or one of the solvates thereof, obtained by the process according to the invention as defined above.

### Composition Comprising the Compound of Formula (I) or (I′)

Another subject of the present invention is a composition comprising at least one compound of formula (I) or (I′), preferably (I″), and/or one of the salts thereof with an organic or inorganic acid or base, and/or one of the geometrical or optical isomers thereof and/or one of the solvates thereof such as hydrates, obtained by the process according to the invention as described above.

Preferably, the composition comprises at least one compound of formula (I) or (I′), preferably (I″), and/or one of the salts thereof, and/or one of the geometrical or optical isomers thereof and/or one of the solvates thereof, in a content of greater than 10% by weight, relative to the total weight of the composition.

For the purposes of the present invention, “an amount or a content ofgreater than 10% by weight” is understood to mean that the compound of formula (I) or (T′) is present in an amount or a content of strictly greater than 10% by weight, relative to the total weight of the composition, that is to say that the value of 10% by weight is excluded.

If the composition of the invention comprises several different compounds of formula (I) or (I′) “an amount or a content of greater than 10% by weight” is understood to mean that the sum, by weight, of the compounds of formula (I) or (I′) is strictly greater than 10% by weight, relative to the total weight of the composition, that is to say that the value of 10% by weight is excluded.

Preferably, the compound(s) of formula (I) or (I′), obtained according to the process as defined above, is(are) present in a content of greater than or equal to 20% by weight, more preferentially in a content of greater than or equal to 30% by weight, even more preferentially in a content of greater than or equal to 40% by weight, better still in a content of greater than or equal to 50% by weight, even better still in a content of greater than or equal to 60% by weight, in particular in a content of greater than or equal to 70% by weight, more particularly in a content of greater than or equal to 80% by weight, notably in a content of greater than or equal to 90% by weight, relative to the total weight of the composition according to the invention.

Preferably, the compound of formula (I″), obtained according to the process as defined above, is present in a content of greater than or equal to 20% by weight, more preferentially in a content of greater than or equal to 30% by weight, even more preferentially in a content of greater than or equal to 40% by weight, better still in a content of greater than or equal to 50% by weight, even better still in a content of greater than or equal to 60% by weight, in particular in a content of greater than or equal to 70% by weight, more particularly in a content of greater than or equal to 80% by weight, notably in a content of greater than or equal to 90% by weight, relative to the total weight of the composition according to the invention.

Preferably, the compound(s) of formula (I) or (I′), obtained according to the process as defined above, is(are) present in a content ranging from 30% to 99% by weight, more preferentially in a content ranging from 50% to 98% by weight, even more preferentially in a content ranging from 60% to 97% by weight, better still in a content ranging from 70% to 96% by weight, even better still in a content ranging from 75% to 95% by weight, in particular in a content ranging from 80% to 94% by weight, more particularly in a content ranging from 85% to 93% by weight, such as 90% by weight, relative to the total weight of the composition.

Preferably, the compound of formula (I″), obtained according to the process as defined above, is present in a content ranging from 30% to 99% by weight, more preferentially in a content ranging from 50% to 98% by weight, even more preferentially in a content ranging from 60% to 97% by weight, better still in a content ranging from 70% to 96% by weight, even better still in a content ranging from75% to 95% by weight, in particular in a content ranging from 80% to 94% by weight, more particularly in a content ranging from 85% to 93% by weight, such as 90% by weight, relative to the total weight of the composition.

Advantageously, the compound(s) of formula (I) or (I′), preferably the compound of formula (I″), obtained according to the process as defined above, is (are) present in a content ranging from 75% to 95% by weight, in particular in a content ranging from 80% to 94% by weight, more particularly in a content ranging from 85% to 93% by weight, such as 90% by weight, relative to the total weight of the composition.

Advantageously, the compound of formula (I″), obtained according to the process as defined above, is present in a content ranging from 75% to 95% by weight, in particular in a content ranging from 80% to 94% by weight, more particularly in a content ranging from 85% to 93% by weight, such as 90% by weight, relative to the total weight of the composition.

Preferably, the composition further comprises at least one (non)polar (a)protic organic solvent, preferably polar protic organic solvent.

Preferably, the (non)polar (a)protic (a)protic organic solvent(s) is (are) chosen from polar protic organic solvents such as polyols.

The term “(poly)ol” is understood to mean a compound which is liquid at room temperature (25° C.) and at atmospheric pressure (1.013×10⁵ Pa) comprising a hydrocarbon chain of between C₂ and C₁₀, particularly between C₂ and C₅, more particularly between C₃ and C₄, such as C₃, which is linear or branched, saturated or unsaturated, preferably saturated and comprising one or more hydroxyl groups, preferably comprising between 2 and 5 hydroxyl groups, more particularly comprising between 2 and 3, more preferentially 2 hydroxyl groups.

Preferably, the (poly)ol(s) is (are) chosen from (C₂-C₁₀)alkane(poly)ols, more preferentially chosen from (C₂-C₆)alkane(poly)ols.

For the purposes of the present invention, the term “(C₂-C₁₀)alkane(poly)ol” is understood to mean a linear or branched, preferably linear, alkane comprising from 1 to 10 carbon atoms and comprising one or more hydroxyl (—OH) groups, preferably 2 or 3 hydroxyl groups, more preferentially 2 hydroxyl groups.

In particular, the (non)polar (a)protic organic solvent(s) is (are) a (C₂-C₆)alkanediol or (C₂-C₆)alkanetriol, preferably (C₂-C₆)alkanediol. More preferentially, the (non)polar (a)protic organic solvent(s) is (are) a (C₂-C₄)alkanediol or (C₂-C₄)alkanetriol, preferably (C₂-C₄)alkanediol.

Preferably, the (non)polar (a)protic organic solvent is propanediol, in particular chosen from the group constituted of propane-1,2-diol (or propylene glycol), propane-1,3-diol, butane-1,3-diol (or butylene glycol) and mixtures thereof, preferably 1,3-propanediol.

According to a particular embodiment of the invention, the composition comprises, as (non)polar (a)protic organic solvent, one or more (C₂-C₆)alkanols, preferably (C₂-C₄)alkanols such as ethanol.

According to a particular embodiment of the invention, the composition comprises a mixture of (non)polar (a)protic organic solvents different from one another, in particular a mixture of one or two (C₂-C₆)alkanediols, preferably two (C₂-C₄)alkanediols with a (C₂-C₆)alkanol, preferably (C₂-C₄)alkanol such as ethanol.

According to another particular embodiment of the invention, the composition comprises a mixture of (non)polar (a)protic organic solvents different from one another: two or more (C₂-C₆)alkanediols, preferably two (C₂-C₄)alkanediols.

Preferably, the (non)polar (a)protic organic solvent(s) can be present in an amount of greater than or equal to 1% by weight, particularly in an amount of greater than or equal to 9% by weight, more particularly in an amount of between 2% and 75%, even more particularly between 3% and 50% by weight, preferably in an amount ranging from 3% to 20% by weight, more preferentially in an amount ranging from 5% to 15% by weight, such as 9% by weight, relative to the total weight of the composition.

According to one embodiment of the invention, the composition further comprises ii) at least one (non)polar (a)protic organic solvent such as at least one polyol as defined above in an amount of less than or equal to 70% by weight, particularly in an amount of less than or equal to 50%, preferably in an amount of less than or equal to 20% by weight, preferably in an amount of between 1% and 15%, more preferentially between 5% and 12% such as 9% by weight relative to the total weight of the composition.

Advantageously, the composition comprises i) at least one compound of formula (I) or (I′), preferably of formula (I″), obtained according to the process according to the invention, in an amount of greater than 10% by weight, relative to the total weight of the composition, and ii) at least one polar protic organic solvent, preferably at least one (C₂-C₆)alkane(poly)ol.

Advantageously, the composition comprises at least one compound of formula (I) or (I′), preferably of formula (I″), obtained according to the process according to the invention, in an amount of greater than 10% by weight, relative to the total weight of the composition, and at least one (C₂-C₆)alkanepolyol, preferably at least one (C₂-C₆)alkanediol.

Advantageously, the composition comprises a content of more than 10% by weight of one or more compounds of formula (I) or (I′), preferably of formula (I″), obtained according to the process according to the invention, and at least one polar protic organic solvent chosen from the group constituted of propane-1,2-diol, propane-1,3-diol and mixtures thereof, more preferentially 1,3-propanediol.

Preferably:
the compound of formula (I) or (I′), preferably of formula (I″), obtained according to the process according to the invention, is present in a content ranging from 30% to 99% by weight, more preferentially in a content ranging from 50% to 98% by weight, even more preferentially in a content ranging from 6% to 97% by weight, better still in a content ranging from 70% to 96% by weight, even better still in a content ranging from75% to 95% by weight, in particular in a content ranging from 80% to 94% by weight, more particularly in a content ranging from 85% to 93% by weight, such as 90% by weight, relative to the total weight of the composition, and
the (non)polar (a)protic organic solvent, preferably polar protic organic solvent, is present in an amount of greater than or equal to 1% by weight, particularly in an amount of greater than or equal to 9% by weight, more particularly in an amount of between 2% and 75%, even more particularly between 3% and 50% by weight, preferably in an amount ranging from 3% to 20% by weight, more preferentially in an amount ranging from 5% to 15% by weight, such as 9% by weight, relative to the total weight of the composition.

Preferably:
the compound of formula (I) or (I′), preferably of formula (I″), obtained according to the process according to the invention, is present in a content ranging from 30% to 99% by weight, more preferentially in a content ranging from 50% to 98% by weight, even more preferentially in a content ranging from 60% to 97% by weight, better still in a content ranging from 70% to 96% by weight, even better still in a content ranging from 75% to 95% by weight, in particular in a content ranging from 80% to 94% by weight, more particularly in a content ranging from 85% to 93% by weight, such as 90% by weight, relative to the total weight of the composition, and
the (non)polar (a)protic organic solvent, preferably polar protic organic solvent, is present in an amount of less than or equal to 70% by weight, particularly in an amount of less than or equal to 50%, preferably in an amount of less than or equal to 20% by weight, preferably in an amount of between 1% and 15%, more preferentially between 5% and 12% such as 9% by weight relative to the total weight of the composition.

Preferably, the composition according to the invention comprises at least one compound of formula (I) or (I′), preferably of formula (I″), and/or one of the salts thereof, and/or one of the geometrical or optical isomers thereof and/or one of the solvates thereof, obtained by the process according to the invention as defined above, at least one (non)polar (a)protic organic solvent, preferably polar protic organic solvent, and optionally water, preferably water.

Preferably, the composition according to the invention comprises at least one compound of formula (I) or (I′), preferably of formula (I″), and/or one of the salts thereof, and/or one of the geometrical or optical isomers thereof and/or one of the solvates thereof, at least one (non)polar (a)protic organic solvent, preferably polar protic organic solvent, and optionally water, preferably water.

Preferably, the water may be present in a content of less than or equal to 20% by weight, preferably less than or equal to 10% by weight, more preferentially in a content ranging from 0.05% to 5% by weight, even more preferentially in a content ranging from 0.5% to 3% by weight, better still in a content ranging from 0.8% to 1.5% by weight, such as 1% by weight, relative to the total weight of the composition.

Advantageously, the composition according to the invention comprises at least one compound of formula (I) or (I′), preferably of formula (I″), obtained according to the process according to the invention, in an amount of greater than 10% by weight, relative to the total weight of the composition, at least one (non)polar (a)protic organic solvent, preferably polar protic organic solvent, in an amount of greater than or equal to 1% by weight, relative to the total weight of the composition, and water in a content of less than or equal to 20% by weight, relative to the total weight of the composition.

Advantageously,
the compound of formula (I) or (I′), preferably of formula (I″), obtained according to the process as described above, is present in a content ranging from 75% to 95% by weight, relative to the total weight of the composition,
the (non)polar (a)protic organic solvent, preferably polar protic organic solvent, is present in an amount ranging from 5% to 20% by weight, relative to the total weight of the composition,
the water may be present in a content ranging from 0.05% to 5% by weight relative to the total weight of the composition.

Advantageously,
the compound of formula (I) or (I′), preferably of formula (I″), is present in a content ranging from 75% to 95% by weight, relative to the total weight of the composition,
the (non)polar (a)protic organic solvent, preferably polar protic organic solvent, is present in an amount ranging from 5% to 20% by weight, relative to the total weight of the composition,
the water may be present in a content ranging from 0.05% to 5% by weight relative to the total weight of the composition.

More advantageously, the composition consists of:
i) 75% to 95% by weight of compound(s) of formula (I) or (I′), preferably of formula (I″), obtained according to the process as defined above, relative to the total weight of the composition,
ii) 5% to 20% by weight of (non)polar (a)protic organic solvent(s), preferably polar protic organic solvent(s), relative to the total weight of the composition, and
iii) 0.05% to 5% by weight of water, relative to the total weight of the composition.

More advantageously, the composition consists of:
i) 75% to 95% by weight of compound(s) of formula (I) or (I′), preferably of formula (I″), relative to the total weight of the composition,
ii) 5% to 20% by weight of (non)polar (a)protic organic solvent(s), preferably polar protic organic solvent(s), relative to the total weight of the composition, and
iii) 0.05% to 5% by weight of water, relative to the total weight of the composition.

Better still, the composition of the invention consists of:
i) 75% to 95% by weight of a compound of formula (I) or (I′), preferably of formula (I″), obtained according to the process as defined above, relative to the total weight of the composition,
ii) 5% to 20% by weight of a (non)polar (a)protic organic solvent, preferably polar protic organic solvent, preferably a (C₂-C₆)alkanediol, more preferentially chosen from propane-1,2-diol, propane-1,3-diol and mixtures thereof, even more preferentially 1,3-propanediol, relative to the total weight of the composition, and
iii) 0.05% to 5% by weight of water, relative to the total weight of the composition.

Better still, the composition of the invention consists of:
i) 75% to 95% by weight of a compound of formula (I) or (I′), preferably of formula (I″), relative to the total weight of the composition,
ii) 5% to 20% by weight of a (non)polar (a)protic organic solvent, preferably polar protic organic solvent, preferably a (C₂-C₆)alkanediol, more preferentially chosen from propane-1,2-diol, propane-1,3-diol and mixtures thereof, even more preferentially 1,3-propanediol, relative to the total weight of the composition, and
iii) 0.05% to 5% by weight of water, relative to the total weight of the composition.

According to a preferred embodiment of the invention, the composition consists of i) 90%+/−5% by weight of compound of formula (I) or (I′), preferably of formula (I″), obtained according to the process according to the invention as described above, ii) 9%+/−2% by weight of polar protic organic solvent, notably of polyol, preferably a (C₂-C₆)alkanediol, more preferentially selected from the group constituted of propane-1,2-diol, propane-1,3-diol, butylene glycol and mixtures thereof, even more preferentially 1,3-propanediol, and 1%+/−0.5% by weight of water, provided that the sum of the ingredients i)+ii)+iii) is equal to 100%.

According to a preferred embodiment of the invention, the composition consists of i) 90%+/−5% by weight of compound of formula (I) or (I′), preferably of formula (I″), ii) 9%+/−2% by weight of polar protic organic solvent, notably of polyol, preferably a (C₂-C₆)alkanediol, more preferentially selected from the group constituted of propane-1,2-diol, propane-1,3-diol, butylene glycol and mixtures thereof, even more preferentially 1,3-propanediol, and 1%+/−0.5% by weight of water, provided that the sum of the ingredients i)+ii)+iii) is equal to 100%.

According to a preferred embodiment of the invention, the composition consists of i) 90%+/−5% by weight of compound of formula (I) or (I′), preferably of formula (I″), obtained according to the process as defined above, ii) 9%+/−2% by weight of polar protic organic solvent, notably of polyol, preferably a (C₂-C₆)alkanediol, more preferentially selected from the group constituted of propane-1,2-diol, propane-1,3-diol, butylene glycol and mixtures thereof, even more preferentially 1,3-propanediol, and 1%+/−0.5% by weight of water, provided that the sum of the ingredients i)+ii)+iii) is equal to 100%.

According to a preferred embodiment of the invention, the composition consists of i) 90%+/−5% by weight of compound of formula (I) or (I′), preferably of formula (I″), ii) 9%+/−2% by weight of polar protic organic solvent, notably of polyol, preferably a (C₂-C₆)alkanediol, more preferentially selected from the group constituted of propane-1,2-diol, propane-1,3-diol, butylene glycol and mixtures thereof, even more preferentially 1,3-propanediol, and 1%+/−0.5% by weight of water, provided that the sum of the ingredients i)+ii)+iii) is equal to 100%.

According to a more preferred embodiment of the invention, the composition consists of i) 90% by weight of compound of formula (I″) obtained according to the process as defined above, ii) 9% by weight of 1,3-propanediol, as substance which is liquid at room temperature and atmospheric pressure, and 1% by weight of water relative to the total weight of the composition.

According to a more preferred embodiment of the invention, the composition consists of i) 90% by weight of compound of formula (I″), ii) 9% by weight of 1,3-propanediol, and 1% by weight of water relative to the total weight of the composition.

### Compound of Formula (IV′)

The present invention also relates to at least one compound of formula (IV′) below, the reduced form (IV′red) thereof, and also one of the optical isomers or Z/Z, Z/E, E/Z and E/E geometrical isomers thereof, and/or one of the salts thereof with an organic or inorganic acid or base, and/or one of the solvates thereof, such as the hydrates:

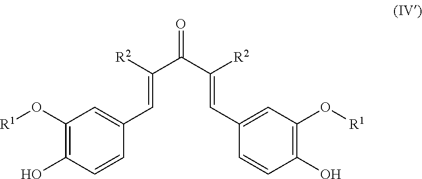

in which formula (IV′):
   R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
   R² represents a hydrogen atom or a C₂-C₄ alkyl group, such as methyl or ethyl; preferably R² represents a hydrogen atom.

Preferably, R¹ represents a linear or branched, saturated or unsaturated hydrocarbon group comprising from 2 to 6 carbon atoms.

Preferably, R¹ represents a C₂-C₄ alkyl group and R² represents a hydrogen atom or a C₂-C₄ alkyl group, such as ethyl; preferably R² represents a hydrogen atom.

Preferably, when the compound (IV′) comprises R¹ representing an ethyl group, and R² a hydrogen atom, then the compound (IV′) is in the form of a salt of an inorganic base.

The reduced form of (IV′) is understood to mean the compound of formula (IV′red) below, one of the salts thereof with an organic or inorganic acid or base, and/or one of solvates thereof, such as the hydrates:

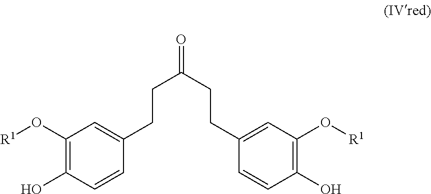

Preferably, R¹ represents a linear or branched, saturated or unsaturated hydrocarbon group comprising from 2 to 6 carbon atoms.

Preferably, R¹ represents a C₂-C₄ alkyl group and R² represents a hydrogen atom or a C₂-C₄ alkyl group, such as ethyl; preferably R² represents a hydrogen atom.

Preferably, in formula (IV′), R¹ represents a C₁-C₄ alkyl group, more preferably a C₂-C₄ alkyl group, such as ethyl, R² represents a hydrogen atom.

Furthermore, the present invention also relates to a composition comprising at least one compound of formula (IV′), the reduced form (IV′red) thereof, and also one of the optical isomers or Z/Z, Z/E, E/Z and E/E geometrical isomers thereof, and/or one of the salts thereof with an organic or inorganic acid or base, and/or one of the solvates thereof, such as the hydrates.

The compound(s) of formulae (I), (I′), (I″) or (IV′), as defined above, may in particular be used in a composition comprising a physiologically acceptable medium.

The physiologically acceptable medium, in which the compounds of formulae (I), (I′), (I″) or (IV′) can be used, may be chosen by those skilled in the art on the basis of their general knowledge depending on the type of composition desired.

The invention is illustrated in more detail in the following non-limiting examples.

### EXAMPLE 1

### Step 1: Preparation of the Compound of Formula (IV)

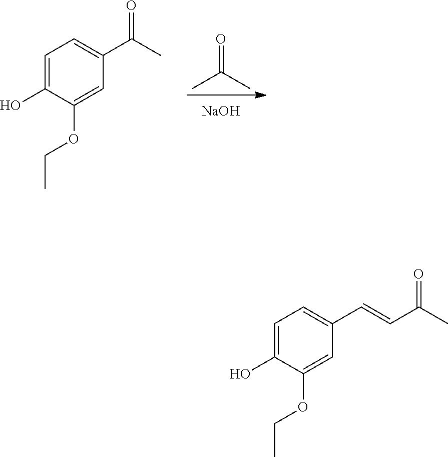

Ethylvanillin (300 g, 1.81 mol) and then water (1140 ml, 3.8 V) at a temperature of 20° C. are introduced into a reactor. The suspension is stirred before loading the acetone (600 ml, 2 V). The medium is cooled to 15° C. and then the 50% sodium hydroxide solution (400 ml, 7.6 mol) is run in over 1 hour without exceeding 25° C. The medium is then heated at 30° C. for 12 h. It turns red and the phenate precipitates. The suspension is cooled to a temperature of 5° C. and then filtered under pressure. The filter cake is washed with acetone at 5° C. (578 ml, 1.9 V) and then with water (227 ml, 0.8 V). The wet solid is reloaded into a reactor in the presence of water (1755 ml, 5.9 V) and acetone (672 ml, 2.2 V). 33% hydrochloric acid is run into this suspension at a temperature of 20° C. over 1 hour while maintaining the temperature of the medium at 15° C. A previously prepared mixture of hydrochloric acid (115 ml, 1.23 mol), water (130 ml, 0.43 V) and acetone (48 ml, 0.16 V) is run into the solution obtained.

The pH is preferably less than 4. The suspension is stirred for two hours at a temperature of 25° C. and then cooled to a temperature of 5° C. before being filtered under pressure.

The filter cake is washed twice with water (2×377 ml, 2×1.3 V) then centrifuged until a solids content >35% is obtained. The yield is of the order of 90% and provides a product with an HPLC titre >97%.

### Step of Dissolving the Compound of Formula (IV)

The water-wetted filter cake (770 g, 1.57 mol) is introduced into a reactor with methyl-THF (1041 ml, 1.35 V) and water (74 ml, 0.1 V). A two-phase solution is obtained. The pH is preferably greater than 5.

The two-phase solution is heated to a temperature of 45° C. and is then cooled to a temperature of 20° C. before decanting the mixture and drawing off the aqueous phase. The organic phase is washed again with water at 20° C. (148 ml, 0.2 V) which is then decanted and the water is removed by distillation at 82-85° C. using Dean-Stark apparatus. The medium is then concentrated to reach a solids content of 20-28%. The dry estimated yield is greater than 95% and the HPLC purity is greater than 97%.

### Step 2: Catalytic Hydrogenation to Obtain the Compound of Formula (I′)

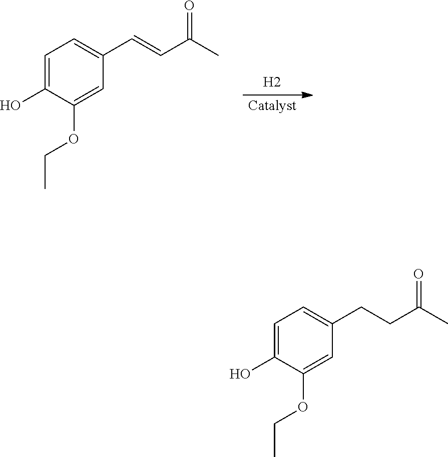

The methyl-THF solution (1199 ml, 1.57 mol) is introduced into a hydrogenator with 5% palladium on charcoal containing 50% water (1.62 g, 0.14% w/w) and sodium (bi)carbonate.

The hydrogen is then introduced and the reaction medium is heated to a temperature in a range of from 100° C. to 150° C. The hydrogenation reaction is carried out until all the hydrogen has been consumed.

The reaction medium is then cooled to a temperature of 40° C. before filtering off the catalyst. The reactor and the catalyst are rinsed with methyl-THF (100 ml, 0.1 V). This filtrate is added to the first filtrate in order to be used in the purification. The dry estimated yield is greater than 95% and the HPLC purity is greater than 95%.

### Acid-Base Washing Step

Introduced into a reactor at a temperature of 20° C. are: the solution containing the compound of formula (I) (2697 g, 2.98 mol) obtained during the catalytic hydrogenation step, water (85 ml, 0.03 V) and sodium hydroxide (1.9 ml) to pH 12.

The reaction medium is stirred for a period of 10 minutes before drawing off the aqueous phase. The organic phase is washed again with water (85 ml) which is then decanted.

The content of ethylvanillin in the organic phase is checked (≤0.4% HPLC) and then water (85 ml) and 33% hydrochloric acid (1.2 ml) are added to pH 1.5-2. The medium is stirred for 10 minutes before drawing off the aqueous phase. The organic phase is then washed again with water (85 ml) which is then decanted and drawn off and then the methyl-THF is removed by distillation under vacuum at 80° C. until a solids content of at least 97% is reached. The crude product is isolated with 90% yield in the form of a thick honey at 50° C. with a purity of around 98%.

### Short-Path Distillation Step

On conclusion of the acid-base washing steps, the compound of formula (I) is melted and loaded into a feed tank of a distillation apparatus at 50° C. and is introduced at a constant flow rate into a short-path distillation apparatus.

The crude product is isolated with 90% yield in the form of a thick honey at 50° C. with a purity of around 98%.

### Dilution Step

The premelted compound of formula (I) is loaded into a reactor at 50° C. with a polar protic organic solvent, preferably a (C₁-C₆)alkane(poly)ol and water.

After stirring for 30 minutes at 50° C., the product is packaged with a yield of 100%.

### EXAMPLE 2

Step 1: Preparation of the Compound of Formula (IV′a) and the Salt Thereof with an Inorganic Base (Sodium Hydroxide).

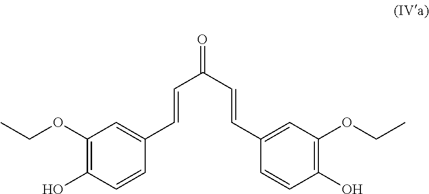 [SMILES: CCOc1cc(/C=C/C(=O)/C=C/c2ccc(O)c(OCC)c2)ccc1O]

Ethylvanillin (1.717 g), acetone (0.3 g) and then EtOH (3.16 g) are introduced into a reactor. The reaction mixture is kept for a few minutes at 45° C. until said mixture becomes homogeneous. A 50% solution containing 2.7 g of sodium hydroxide (2.7 g) and 2.7 g of water is then introduced over a few minutes until a white suspension is formed which then changes from white to yellow, and then the reaction mixture becomes homogeneous and reddish. The mixture is then left stirring for 48 h at room temperature (25° C.) until the mixture turns blood red. The compound of formula (IV′a) is then obtained in the form of a sodium salt (which can be isolated by filtration for example).

A pH-meter probe is then introduced into the reaction mixture comprising (IV′a) in the form of a salt, the pH of which is around 12.9. The latter is neutralized by adding an organic or inorganic acid such as HCl aq (concentration approximately 4M) to a pH close to 7 (pH=7.2). Next, 20 ml of ethyl acetate (EtOAc) are added, and then the mixture is transferred to a separating funnel (stirring, decantation), and the aqueous phase is extracted with 2×EtOAc (2×20 ml), followed by drying over MgSO₄, filtration and concentration under vacuum. Optionally, the crude product can be purified by chromatography on silica gel: 80 g column Eluent: 80:20 Heptane/EtOAc to 60:40. An orange solid is isolated corresponding to compound (Iv′a), the chemical structure of which was confirmed by conventional spectroscopic and spectrometric methods.

Step 2: The Compound (IV′a) can then be Hydrogenated According to the Same Conditions as Step 2) of Example 1 in Order to Yield the Compound (IV′a-Reduced).

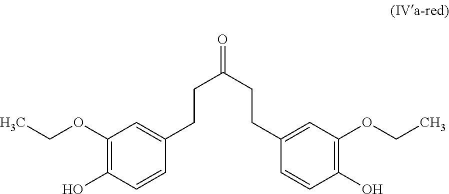 [SMILES: CCOc1cc(CCC(=O)CCc2ccc(O)c(OCC)c2)ccc1O]

1. Process for preparing at least one compound of formula (I) below: 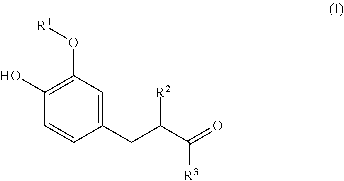
in which formula (I):
R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
R² represents a hydrogen atom or a C₂-C₄ alkyl group, such as methyl or ethyl; preferably R² represents a hydrogen atom;
R³ represents a linear or branched, saturated or unsaturated hydrocarbon group comprising from 1 to 12 atoms, optionally substituted with an aryl group; the aryl group preferably being a phenyl group optionally substituted with one or more groups chosen from hydroxyl, C₁-C₄ alkyl group, C₁-C₄ alkoxy group and combinations thereof; preferably R³ represents a C₁-C₆ alkyl group; preferably a C₁-C₄ alkyl group;
and also one of the optical isomers or geometrical isomers thereof, and/or one of the salts thereof with an organic or inorganic acid or base, and/or one of the solvates thereof, such as the hydrates;
said process taking place according to the following synthesis scheme (1): 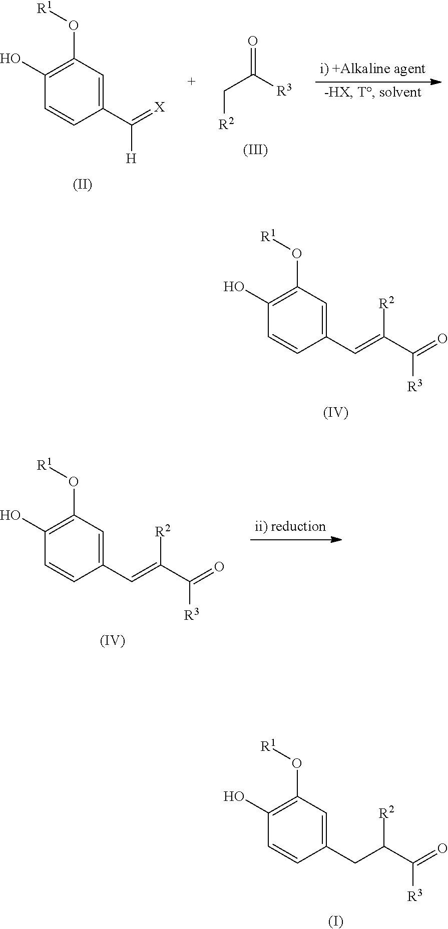
in which synthetic pathway (1):
X represents a heteroatom chosen from oxygen and sulfur, preferably an oxygen atom,
R¹, R² and R³ have the same meanings as in the formula (I);
said process being characterised in that it comprises:
at least one step (i) of reacting the compound of formula (II) in the presence of:
at least one compound of formula (III),
at least one (non)polar (a)protic solvent, and
at least one alkaline agent in an excess molar amount relative to the compound of formula (II),
in order to result in the formation of at least one precipitate including at least one compound of formula (IV), geometrical isomers thereof and/or one of the one of the salts thereof with an organic or inorganic base; and
at least one step (i1) of separating the precipitate from the reaction medium of step (i), then
at least one step (i2) of acidifying the precipitate using at least one inorganic or organic, preferably inorganic acidifying agent, before carrying out the reaction step (ii).

2. Process according to claim 1, characterized in that the alkaline agent(s) of step (i) is or are inorganic alkaline agents chosen from the group constituted of alkali metal or alkaline earth metal hydroxides, alkali metal or alkaline earth metal (bi)carbonates, and mixtures thereof, in particular alkali metal or alkaline earth metal hydroxides, in particular sodium hydroxide.

3. Process according to claim 1, characterized in that step (i) comprises the reaction of:
at least one compound of formula (II),
preferably, in formula (II), X represents an oxygen atom,
in the presence of:
at least one compound of formula (III),
preferably, in formula (III), R² represents a hydrogen atom and R³ represents a C₁-C₆ alkyl group, preferably a C₁-C₄ alkyl group, preferably a methyl or ethyl group;
advantageously, said compound of formula (III) is present in the reaction medium of step (i) in an amount less than or equal to 5 molar equivalents, which makes it possible to improve the kinetics of reaction step (i), more preferentially in an amount ranging from 2 to 5 molar equivalents, better still in an amount ranging from 2.5 to 5 molar equivalents, in particular in an amount of 4.5 molar equivalents, relative to the compound of formula (II);
at least one (non)polar (a)protic solvent, preferably at least one polar protic solvent, such as water,
preferably, as a mixture with at least one (non)polar (a)protic organic solvent, preferably (non)polar aprotic organic solvent, such as nonpolar aprotic organic solvents, preferably having a dielectric constant E ranging from 1 to 11 and a dipole moment p ranging from 0 to 2, in particular those chosen from the group constituted of n-hexane, cyclohexane, 1,4-dioxane, carbon tetrachloride (CCl₄), benzene, tetrachloroethylene (Cl₂C═Cl₂C), toluene, carbon disulfide (CS₂), trichloroethylene (Cl₂C═CHCl), diethyl ether (Et₂O), ethyl acetate (CH₃C(O)OEt or AcOEt), dimethyl ether (DME), tetrahydrofuran (THF), methyltetrahydrofuran (2-MeTHF), dichloromethane (CH₂Cl₂), dichloroethane (C₁CH₂CH₂C₁) and mixtures thereof;
at least one organic or inorganic, preferably inorganic alkaline agent,
more preferentially chosen from the group constituted of alkali metal or alkaline earth metal hydroxides, alkali metal or alkaline earth metal (bi)carbonates, and mixtures thereof, in particular alkali metal or alkaline earth metal hydroxides, in particular sodium hydroxide;
said alkaline agent being in an excess molar amount relative to the compound of formula (II),
preferably, the alkaline agent is in an amount greater than or equal to 2 molar equivalents, more preferentially ranging from 2 to 5 molar equivalents, for example 4.5 molar equivalents, relative to the compound of formula (II).

4. Process according to claim 1, characterized in that the reaction step (i) is carried out at a temperature below or equal to 40° C., preferably at a temperature ranging from 20° C. to 35° C., more preferentially at a temperature ranging from 25° C. to 35° C., in particular at a temperature of 30° C.+/−1° C.

5. Process according to claim 1, characterized in that, once the reaction step (i) is completed, the reaction medium is cooled to a temperature below or equal to 25° C., preferably in a temperature range of from 1° C. to 12° C., preferably ranging from 3° C. to 8° C.

6. Process according to claim 1, characterized in that the step (i1) of separating said precipitate from the reaction medium of step (i) is carried out by filtration, preferably by pressure filtration.

7. Process according to claim 1, characterized in that the acidification step (i2) is carried out with the aid of at least one inorganic acidifying agent, preferably of H⁺Hal⁻ type, with Hal representing a halogen atom chosen from the group constituted of chlorine, bromine and iodine, more preferentially, the acidifying agent is hydrochloric acid.

8. Process according to claim 1, characterized in that, after steps (i1) and (i2) have been carried out, the compound of formula (IV) is dissolved in at least one (non)polar aprotic organic solvent, preferably in at least one nonpolar aprotic organic solvent, optionally as a mixture with at least one polar protic solvent, such as water, before step (ii) is carried out.

9. Process according to claim 8, the pH of the reaction medium, when dissolving the compound of formula (IV) with at least one (non)polar aprotic organic solvent, is greater than 5.

10. Process according to claim 1, characterized in that step (ii) is a reduction by catalytic hydrogenation or a reduction by reducing agent chosen from alkali metal hydrides such as alkali metal borohydrides such as NaBH₄, alkali metal aluminium tetrahydrides such as LiAlH₄, preferably the catalyst is present in the reaction medium of step (ii) in an amount ranging from 0.05% to 10%, preferably in a content ranging from 0.1% to 10% by weight, preferably in a content ranging from 0.5% to 1% relative to the total weight of the reaction medium of step (ii).

11. Process according to claim 1, characterized in that step (ii) is a reduction by catalytic hydrogenation, preferably with palladium (Pd), for example on carbon (Pd/C), in particular in the presence of at least one alkaline agent having a pKa ranging from 8 to 11, preferably from 9 to 11, notably from 9 to 10, preferably chosen from the group constituted of alkali metal or alkaline earth metal (bi)carbonates and mixtures thereof.

12. Process according to claim 10, characterized in that, on conclusion of step (ii), the compound of formula (I) is purified by short-path distillation.

13. Process according to claim 1, characterized in that in formula (I) R² represents a hydrogen atom and/or R¹ represents a linear or branched, preferably linear C₁-C₄ alkyl group, more preferentially a linear, C₂-C₄ alkyl group, such as ethyl and/or R³ represents a linear or branched C₁-C₆ alkyl group.

14. Process according to claim 1, characterized in that in formula (I), R¹ represents a C₁-C₄ alkyl group, more preferentially a C₂-C₄ alkyl group, such as ethyl; R² represents a hydrogen atom; R³ represents a C₁-C₆ alkyl group; preferably, a C₁-C₄ alkyl group, more preferentially a C₁-C₃ alkyl group, in particular a C₁ alkyl group.

15. Compound of formula (I′) below: 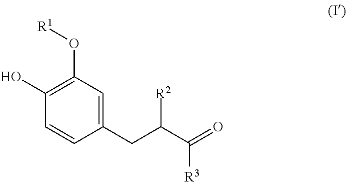
in which formula (I′):
R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
R² represents a hydrogen atom or a C₂-C₄ alkyl group, such as methyl or ethyl; preferably R² represents a hydrogen atom;
R³ represents a branched, saturated or unsaturated hydrocarbon group comprising from 1 to 12 atoms; preferably R³ represents a branched, saturated or unsaturated hydrocarbon group comprising from 3 to 12 carbon atoms; more preferably R³ represents a branched C₁-C₆ alkyl group, more preferentially R³ represents a branched C₃-C₆ alkyl group; especially R³ represents a branched C₃-C₄ alkyl group, in particular a branched C₄ alkyl group;
and also one of the optical isomers or geometrical isomers thereof, and/or one of the salts thereof with an organic or inorganic acid or base, and/or one of the solvates thereof, such as the hydrates.

16. Compound of formula (I) or (I′), and/or one of the optical isomers or geometrical isomers thereof, and/or one of the salts thereof with an organic or inorganic acid or base, and/or one of the solvates thereof, such as the hydrates, obtained according to the process as defined according to claim 1.

17. Composition characterized in that it comprises at least one compound of formula (I) or (I′), and/or one of the optical isomers or geometrical isomers thereof, and/or one of the salts thereof with an organic or inorganic acid or base, and/or one of the solvates thereof, such as the hydrates, obtained according to the process as defined according to claim 1.

18. Composition according to claim 17, characterized in that the compound of formula (I) or (I′) is present in an amount of greater than 10% by weight, relative to the total weight of the composition.

19. Composition according to claim 17, characterized in that it further comprises at least one (non)polar (a)protic organic solvent, preferably polar protic organic solvent, and optionally water.

20. Composition according to claim 19, characterized in that the (non)polar (a)protic organic solvent, preferably polar protic organic solvent, is chosen from the group constituted of (C₂-C₁₀)alkane(poly)ols, preferably (C₂-C₆)alkane(poly)ols.

21. Composition according to claim 17, characterized in that it comprises:
at least one compound of formula (I) or (I′), present in a content ranging from 75% to 95% by weight, relative to the total weight of the composition,
at least one (non)polar (a)protic organic solvent, in an amount ranging from 5% to 20% by weight, relative to the total weight of the composition,
it being possible for water to be present in a content ranging from 0.05% to 5% by weight relative to the total weight of the composition.

22. Composition according to claim 17, characterized in that it consists of i) 90%+/−5% by weight of compound of formula (I) or (I′), ii) 9%+/−2% by weight of polar protic organic solvent in particular a polyol, preferably a (C₂-C₆)alkanediol, more preferentially selected from the group constituted of propane-1,2-diol, propane-1,3-diol, butylene glycol and mixtures thereof, even more preferentially 1,3-propanediol, and 1%+/−0.5% by weight of water, provided that the sum of the ingredients i)+ii)+iii) is equal to 100%.

23. Compound of formula (IV′) below, the reduced form (IV′red) thereof, and also one of the optical isomers or Z/Z, Z/E, L/Z and L/E geometrical isomers thereof, and/or one of the salts thereof with an organic or inorganic acid or base, and/or one of the solvates thereof, such as the hydrates: 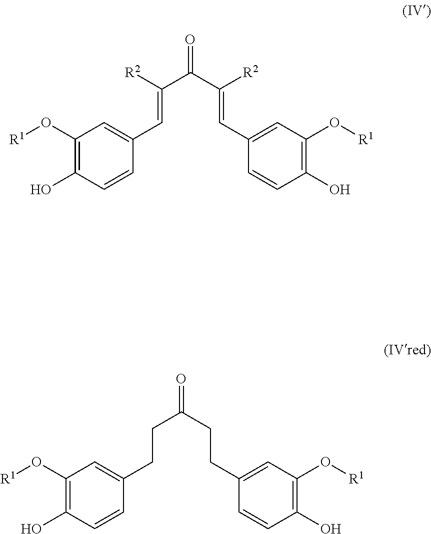
in which formula (IV′) or (IV′red):
R¹ represents a linear or branched, saturated or unsaturated hydrocarbon group comprising from 1 to 6 carbon atoms; preferably R¹ represents a C₁-C₄ alkyl group, more preferentially a C₂-C₄ alkyl group, such as ethyl;
R² represents a hydrogen atom or a C₂-C₄ alkyl group, such ethyl; preferably R² represents a hydrogen atom.

24. Composition comprising, in a physiologically acceptable medium, one or more compounds of formula (IV′), as described according to claim 23, and also the optical isomers or Z/Z, Z/E, E/Z and E/E geometrical isomers thereof, and/or the salts thereof with an organic or inorganic acid or base, and/or the solvates thereof, such as the hydrates.
